(19) United States
(12) Patent Application Publication
(10) Pub. No.: US 2026/0345329 A1
(43) Pub. Date: Nov. 5, 2026

(54) PERCUTANEOUS LEAFLET PLICATION

(71) Applicant: Edwards Lifesciences Corporation, Irvine, CA (US)

(72) Inventors: Nicolas Schleiger, Huntington Beach, CA (US); Bhumica A. Amin, Modesto, CA (US); Arnold Cruz Tuason, Claremont, CA (US); Andrew Charles May, Irvine, CA (US); Sam Shafigh, Mission Viejo, CA (US); Travis Zenyo Oba, Yorba Linda, CA (US); Alana Tyler Stein, Newport Beach, CA (US); Nancy Hoang Khuu, Irvine, CA (US); Cristobal R. Hernandez, Santa Ana, CA (US); Jean-Pierre Michel Rabbah, Tustin, CA (US)

(21) Appl. No.: 19/714,175

(22) Filed: Jun. 19, 2026

Related U.S. Application Data
(63) Continuation of application No. PCT/US2024/059805, filed on Dec. 12, 2024.
(60) Provisional application No. 63/613,606, filed on Dec. 21, 2023.

(51) Int. Cl. A61F 2/24; A61F 2/00

(52) U.S. Cl. CPC A61F 2/2463; A61F 2/2466; A61F 2002/0081; A61F 2220/0008; A61F 2220/0091

(57) ABSTRACT

An implant for treating a heart valve comprising a base portion and a first movable arm extending from the base portion and configured to move independently of the base portion to grasp a leaflet of the heart valve. The implant can be used to treat issues with one or more native leaflets of the heart valve.

### CROSS REFERENCE TO RELATED APPLICATION

This application is a continuation of International Patent Application No. PCT/US 2024/059805, filed Dec. 12, 2024, which claims the benefit of U.S. Patent Application No. 63/613,606, filed on Dec. 21, 2023, the entire disclosures of which are incorporated by reference for all purposes.

### BACKGROUND

The present disclosure relates to the field of medical devices and procedures.

Eliminating and/or tightening excess tissue at a leaflet may be one method for correcting valve issues, e.g., leaflet flail and/or prolapse. Surgical plication is a method in which a physician can eliminate excess leaflet tissue by creating a fold and/or tensioning the leaflet using suture stitching.

### SUMMARY

Some implementations of the present disclosure relate to an implant for treating a heart valve including: a base portion; and a first movable arm extending from the base portion and configured to move independently of the base portion to grasp a leaflet of the heart valve.

In some implementations, the techniques described herein relate to an implant, wherein the base portion has a generally curved form.

In some implementations, the techniques described herein relate to an implant, wherein the first movable arm has a generally curved form that is generally opposite a curvature of the base portion.

In some implementations, the techniques described herein relate to an implant, wherein the base portion includes a network of struts forming one or more cells and wherein the first movable arm is configured to extend through at least one of the one or more cells.

In some implementations, the techniques described herein relate to an implant, wherein the base portion forms a gap between ends of the base portion and wherein the first movable arm is configured to extend between the ends of the base portion.

In some implementations, the techniques described herein relate to an implant, wherein the first movable arm is configured to grasp the leaflet against the base portion.

In some implementations, the techniques described herein relate to an implant, further including a clamp extending approximately in-line with the base portion and forming a gap from the base portion.

In some implementations, the techniques described herein relate to an implant, further including a second movable arm extending from the base portion and configured to grasp the leaflet against the first movable arm.

In some implementations, the techniques described herein relate to an implant, further including one or more paddles extending from the base portion and configured to naturally move towards the first movable arm.

In some implementations, the techniques described herein relate to an implant, further including one or more wires configured to hold the one or more paddles away from the first movable arm.

In some implementations, the techniques described herein relate to a method (e.g., usable on a subject such as a living subject or simulation) including: grasping a leaflet of a heart valve with a first movable arm of an implant, the implant including a first paddle held distally from the first movable arm; twisting the implant to cause folding of the leaflet; and releasing the first paddle to cause the first paddle to grasp the leaflet against the first movable arm.

In some implementations, the techniques described herein relate to a method, wherein the implant further includes a second movable arm, the method further including grasping the leaflet between the first movable arm and the second movable arm.

In some implementations, the techniques described herein relate to a method, wherein the first movable arm includes one or more barbs configured to facilitate grasping of the leaflet.

In some implementations, the techniques described herein relate to a method, wherein the implant further includes a second paddle held distally from the first movable arm, the method further including releasing the second paddle to cause the second paddle to grasp the leaflet.

In some implementations, the techniques described herein relate to a method, further including one or more cords coupled to the first paddle and configured to hold the first paddle distally from the first movable arm.

In some implementations, the techniques described herein relate to a method, further including relaxing the one or more cords to release the first paddle.

In some implementations, the techniques described herein relate to a method, wherein the one or more cords are configured to hold the first paddle at an approximately 90-degree angle relative to the first movable arm.

In some implementations, the techniques described herein relate to a method, wherein the one or more cords are configured to hold the first paddle at an approximately 180-degree angle relative to the first movable arm.

In some implementations, the techniques described herein relate to a method, further including delivering the implant via a delivery tube.

In some implementations, the techniques described herein relate to a method, wherein the delivery tube includes an opening, and the one or more cords extend through the opening.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the disclosure. In addition, various features of different disclosed examples can be combined to form additional examples, which are part of this disclosure. Throughout the drawings, reference numbers may be reused to indicate correspondence between reference elements.

FIG. 1 illustrates an example representation of a heart having various features relevant to certain examples of the present inventive disclosure.

FIG. 2A provides a cross-sectional view of the left ventricle and left atrium of an example heart.

FIG. 2B illustrates a top/surgeon's view looking at the mitral valve of the heart.

FIG. 3A provides an illustration of the heart in a state where functional mitral valve regurgitation (FMR) is present.

FIG. 3B shows a mitral valve where the annulus is dilated and deformed causing mitral regurgitation through the commissure.

FIG. 4 illustrates an example implant configured for attachment to one or more leaflets in accordance with one or more examples.

FIG. 5 illustrates another example implant configured for attachment to one or more leaflets in accordance with one or more examples.

FIG. 6 illustrates another example implant configured for attachment to one or more leaflets in accordance with one or more examples.

FIGS. 7A and 7B illustrate movement of an example implant in accordance with one or more examples.

FIG. 8 illustrates an example implant anchored and/or clipped onto a posterior leaflet of a mitral valve in accordance with one or more examples.

FIG. 9 illustrates an example leaflet repair system configured to percutaneously and/or transcatheter repair and/or assist one or more leaflets in a damaged condition in accordance with one or more examples.

FIG. 10 (FIGS. 10-1, 10-2, 10-3, and 10-4) is a flowchart illustrating steps of an example process for delivering one or more implants in accordance with the present disclosure and/or grasping and/or treating one or more leaflets of a heart valve using the implants.

FIG. 11 (FIGS. 11-1, 11-2, 11-3, 11-4, 11-5, and 11-6) provides certain images depicting various features associated with steps of the example process depicted in FIG. 10.

FIG. 12 illustrates a grasper of an example implant of the present disclosure.

FIG. 13 illustrates an example implant in accordance with one or more examples.

FIG. 14 illustrates an example implant configured to grasp and/or clamp onto one or more leaflets of a heart valve.

### DETAILED DESCRIPTION

The headings provided herein are for convenience only and do not necessarily affect the scope or meaning of the claimed subject matter. The present disclosure relates to systems, devices, and methods to determine access for an anatomical feature based on an analysis of one or more images representing a mineral deposit.

Although certain examples are disclosed below, the subject matter extends beyond the specifically disclosed examples to other alternative examples and/or uses, and to modifications and equivalents thereof. Thus, the scope of the claims that may arise here from is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

### Overview

Systems, apparatuses, devices, methods, etc. for mitigating heart valve regurgitation are described herein. In some implementations, systems, apparatuses, devices, methods, etc. include implants/devices that situate within the valvular annulus and anchor within the annulus and/or nearby vasculature. The systems, apparatuses, devices, methods, etc. can be configured to provide contact pressure onto and/or support to the leaflet region experiencing flail, prolapse, rigidity, etc. In some implementations, systems, apparatuses, devices, methods, etc. capable of compressing onto a leaflet and providing contact pressure onto and/or support to the leaflet region experiencing flail, prolapse, rigidity, etc. are described, e.g., compressive devices, clasps, splints, forms, etc. In some implementations, systems, apparatuses, devices, etc. are described that further anchor to the leaflet annulus or a nearby vasculature. Various examples of methods of delivering and implanting systems, apparatuses, devices, etc. at the site of flail, prolapse, rigidity, etc. are described. An example of where these can be helpful is when used at the posterior leaflet of a mitral valve experiencing flail, prolapse, rigidity, and/or another issue.

The described systems, apparatuses, devices, methods, etc. should not be construed as limiting in any way. The present disclosure is directed toward all novel and nonobvious features and aspects of the various disclosed implementations and applications, alone and in various combinations and sub-combinations with one another. The disclosed systems, apparatuses, devices, methods, etc. are not limited to any specific aspect, feature, or combination thereof, nor do the disclosed systems, apparatuses, devices, methods, etc. require that any one or more specific advantages be present or problems be solved.

The techniques, methods, processes, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Various implementations of systems, devices, examples of prosthetic implants, etc. are disclosed herein, and any combination of the described features, components, and options can be made unless specifically excluded. Likewise, the different constructions and features of devices and systems can be mixed and matched, such as by combining any implant device type/feature, attachment type/feature, site of repair, etc., even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

### Example Heart Anatomy

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves can be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

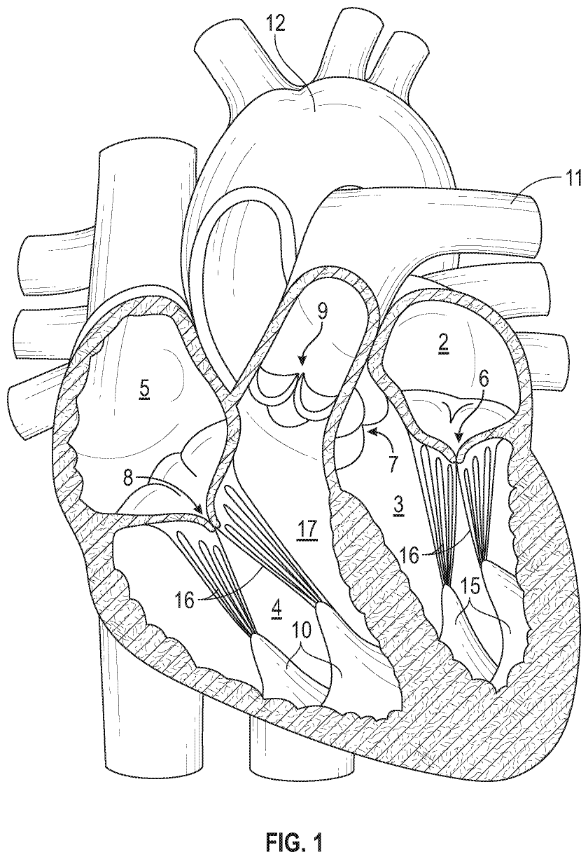

FIG. 1 illustrates an example representation of a heart 1 having various features relevant to certain examples of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The inferior tip 19 of the heart 1 is referred to as the apex (or apex region) and is located on the midclavicular line, in the fifth intercostal space.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11, and can be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The atrioventricular (e.g., mitral and tricuspid) heart valves may further comprise a collection of chordae tendineae and papillary muscles for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve can comprise three leaflets (two shown in FIG. 1) and three corresponding papillary muscles 10 (two shown in FIG. 1). The leaflets of the tricuspid valve may be referred to as the anterior, posterior, and septal leaflets, respectively. The valve leaflets are connected to the papillary muscles 10 by the chordae tendineae 13, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle repositioning disclosed herein are applicable to atrioventricular valves having any number of leaflets and/or papillary muscles associated therewith.

The right ventricular papillary muscles 10 originate in the right ventricle wall, and attach to the anterior, posterior, and septal leaflets of the tricuspid valve, respectively, via the chordae tendineae 13. The papillary muscles 10 of the right ventricle 4 may have variable anatomy; the anterior papillary may generally be the most prominent of the papillary muscles. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

With respect to the mitral valve 6, a normal mitral valve can comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet can comprise a larger surface area in certain anatomies.

The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. The various chords of the chordae tendineae may have different thicknesses, wherein relatively thinner chords are attached to the free leaflet margin, while relatively thicker chords (e.g., strut chords) are attached farther away from the free margin.

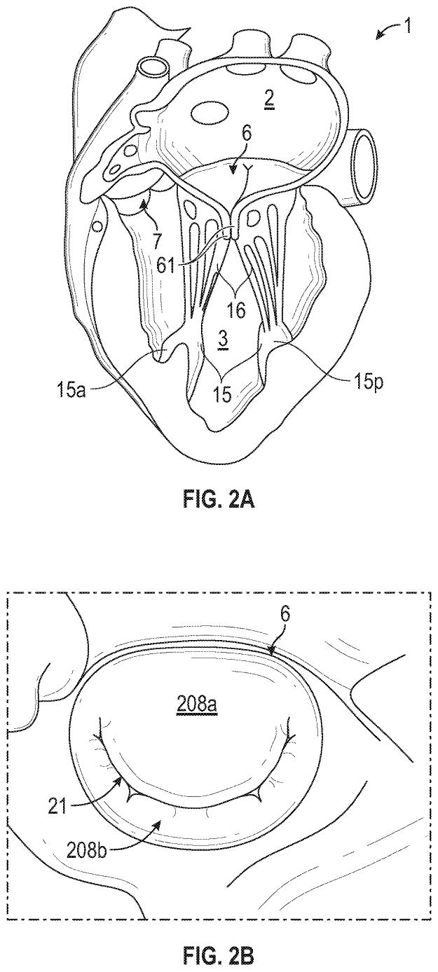

FIG. 2A provides a cross-sectional view of the left ventricle 3 and left atrium 2 of an example heart 1. While some example devices and/or methods are described herein with respect to the left ventricle 3, mitral valve 6, and/or left atrium 2, such devices and/or methods may be applied to and/or performed within other areas of the heart, including the right ventricle, right atrium, and/or tricuspid valve. The diagram of FIG. 2A shows the mitral valve 6, wherein the disposition of the valve 6, papillary muscles 15 and/or chordae tendineae 16 may be illustrative as providing for proper coapting of the valve leaflets to advantageously at least partially prevent regurgitation and/or undesirable flow into the left atrium from the left ventricle 3 and vice versa. Although a mitral valve 6 is shown in FIG. 2A and various other figures provided herewith and described herein in the context of certain examples of the present disclosure, it should be understood that papillary muscle repositioning principles disclosed herein may be applicable with respect to any atrioventricular valve and associated anatomy (e.g., papillary muscles, chordae tendineae, ventricle wall, etc.), such as the tricuspid valve.

As described above, with respect to a healthy heart valve 6 as shown in FIG. 2A, the valve leaflets may extend inward from the valve annulus and come together in the flow orifice to permit flow in the outflow direction (e.g., the downward direction in FIG. 2A) and prevent backflow or regurgitation toward the inflow direction (e.g., the upward direction in FIG. 2A). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae 16 and therefore allowing the chordae tendineae 16 to hold the leaflets in a closed position. The papillary muscles 15 may include a first papillary muscle 15a (e.g., an anterolateral papillary muscle, which may be primarily tethered to the anterior leaflet, for example) and a second papillary muscle 15p (e.g., the posteromedial papillary muscle, which may be primarily tethered to the posterior leaflet, for example). Each of the first papillary muscle 15a and second papillary muscle 15p may provide chordae tendineae 16 to each valve leaflet (e.g., the anterior and posterior leaflets). With respect to the state of the heart 1 shown in FIG. 2A, the proper coaptation of the valve leaflets, which may be due in part to proper position of the papillary muscles 15, may advantageously result in mitral valve operation substantially free of leakage.

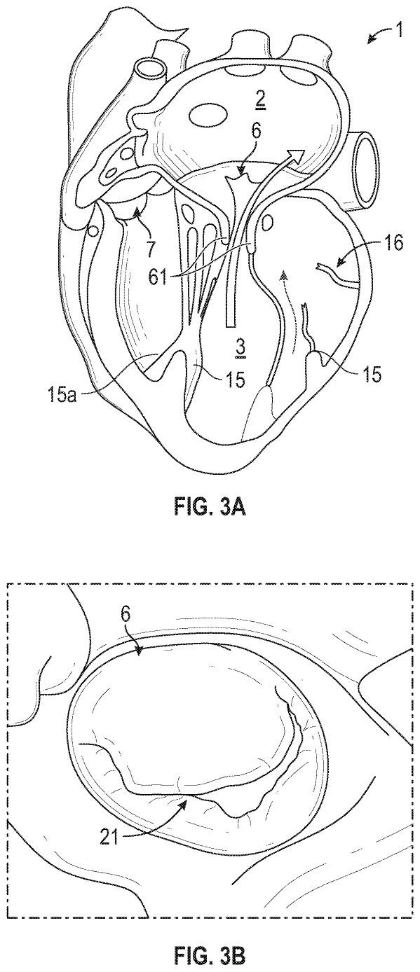

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated. With regard to incompetent heart valves, over time and/or due to various physiological conditions, the position of papillary muscles may become altered, thereby potentially contributing to valve regurgitation. For example, as shown in FIG. 3A, which illustrates a cross-sectional view of a heart 1 experiencing mitral regurgitation flow 20, dilation of the left ventricle may cause changes in the position of the papillary muscles 15 that allow flow 20 back from the ventricle 3 to the atrium 2. Dilation of the left ventricle can be caused by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress, which may eventually result in a dilatory effect that causes papillary muscle alteration resulting in valve dysfunction and degraded cardiac efficiency.

FIG. 2B illustrates a top/surgeon's view looking at the mitral valve 6 of the heart 1. The mitral valve 6 generally includes an anterior leaflet 208a that is relatively large and attaches to the anterior segment of the annulus, while a posterior leaflet 208b is smaller but extends further circumferentially and attaches to the posterior segment of the annulus, as shown in FIG. 2B. The anterior leaflets 208a and the posterior leaflets 208b join and insert into the annulus at the commissures 21, namely the anterior commissure and posterior commissure.

Each of the four valves of the heart has flexible leaflets extending inwardly across the respective orifices that come together or “coapt” in the bloodstream to form the one-way, fluid-occluding surfaces. Accordingly, referring back to the left chambers, oxygenated blood is brought to the left atrium 2 from the pulmonary vein and then transferred across the mitral valve 6 into the left ventricle 3. The left ventricle 3 pumps the oxygenated passing through the aortic valve 7, into the aorta, and throughout the body.

Papillary muscles 15 are attached to the left ventricle 3 wall and connected to the mitral valve 6 leaflets via the chordae tendineae 16. These muscles and cords assist in the function of the mitral valve 6 to open the leaflets to form an aperture, to coapt the leaflets to close the aperture, and to maintain leaflet shape and position.

The coronary sinus is a vasculature that surrounds the left ventricle 3. Throughout the disclosure, the coronary sinus may be used as an example as a nearby vasculature site for docking anchors for various implementations described.

### Example Mitral Valve Conditions

Several diseases can affect the structure and function of the mitral valve 6 and/or other valves. The mitral valve 6 and, less frequently, the tricuspid valve 8, are prone to deformation and/or dilation of the valve annulus, tearing of the chordae tendineae, and/or leaflet prolapse, which results in valvular insufficiency wherein the valve does not close properly and allows for regurgitation or back flow from the left ventricle into the left atrium. Deformations in the structure or shape of the mitral or tricuspid valve can be repairable.

Mitral regurgitation is one of the most common valvular malfunctions in the adult population, and typically involves the elongation or dilation of the posterior two-thirds of the mitral valve 6 annulus, the section corresponding to the posterior leaflet. The most common etiology of systolic mitral regurgitation is myxomatous degeneration, also termed mitral valve prolapse (29% to 70% of cases), which afflicts about 5 to 10 percent of the population in the U.S. Women are affected about twice as often as men. Myxomatous degeneration has been diagnosed as Barlow's syndrome, billowing or ballooning mitral valve, floppy mitral valve, floppy-valve syndrome, prolapsing mitral leaflet syndrome, or systolic click-murmur syndrome. The symptoms include palpitations, chest pain, syncope or dyspnea, and a mid-systolic click (with or without a late systolic murmur of mitral regurgitation). These latter symptoms are typically seen in patients with Barlow's syndrome, where extensive hooding and billowing of both leaflets are the rule. Some forms of mitral valve prolapse seem to be hereditary, though the condition has been associated with Marfan's syndrome, Grave's disease, and other disorders.

Myxomatous degeneration involves weakness in the leaflet structure, leading to thinning of the tissue and loss of coaptation. Barlow's disease is characterized by myxoid degeneration and appears early in life, often before the age of fifty. In Barlow's disease, one or both leaflets of the mitral valve 6 protrude into the left atrium during the systolic phase of ventricular contraction. The valve leaflets are thick with considerable excess tissue, producing an undulating pattern at the free edges of the leaflets. The chordae are thickened, elongated, and may be ruptured. Papillary muscles 15 are occasionally elongated. The annulus is dilated and sometimes calcified. Some of these symptoms are present in other pathologies as well and, therefore, the present application may refer to myxoid degeneration, which is the common pathologic feature of the various diagnoses, including Barlow's syndrome.

Other causes of mitral regurgitation include ischemic heart disease with ischemic mitral regurgitation (IMR), dilated cardiomyopathy (in which the term “functional mitral regurgitation FMR is used), rheumatic valve disease, mitral annular calcification, infective endocarditis, fibroelastic deficiency (FED), congenital anomalies, endocardial fibrosis, and collagen-vascular disorders. IMR is a specific subset of FMR, but both are usually associated with morphologically normal mitral leaflets. Thus, the types of valve disease that lead to regurgitation are varied and present vastly differently.

FIG. 3A provides an illustration of the heart 1 in a state where functional mitral valve regurgitation (FMR) is present. FMR may be considered a disease of the left ventricle 3, rather than of the mitral valve 6. For example, mitral valve regurgitation may occur when the left ventricle 3 of the heart 1 is distorted or dilated, displacing the papillary muscles 15 that support the two valve leaflets 61. The valve leaflets 61 therefore may no longer come together sufficiently to close the annulus and prevent blood flow back into the atrium 2. If left untreated, the FMR experienced in the state shown in FIGS. 3A and 3B may overload the heart 1 and can possibly lead to or accelerate heart failure. Solutions presented herein provide devices and methods for moving the papillary muscles 15 closer to their previous position, which may advantageously reduce the occurrence of mitral regurgitation.

As shown in FIG. 3A, the leaflets 61 of the mitral valve 6 (or tricuspid valve) are not in a state of coaptation, resulting in an opening between the mitral valve leaflets 61 during the systolic phase of the cardiac cycle, which allows the leakage flow 20 of fluid back up into the atrium 2. The papillary muscles 15 may be displaced due to dilation of the left ventricle 3, or due to one or more other conditions, as described above, which may contribute to the failure of the valve 6 to close properly. The failure of the valve leaflets 61 to coapt properly may result in unwanted flow in the outflow direction (e.g., the upward direction in FIG. 3A) and/or unwanted backflow or regurgitation toward the inflow direction (e.g., the downward direction in FIG. 3A).

In some cases, one or more chordae tendineae 16 can rupture, leading to leaflet prolapse and/or flail, as shown in FIG. 3A. Leaflet flail occurs when the coapting portion of the leaflet flips backwards against blood flow. Likewise, leaflet prolapse occurs when a portion of the leaflet protrudes backward. Flail and prolapse can occur due to various conditions, including (but not limited to) papillary muscle 15 and/or chordae tendineae 16 dysfunction. Breaks in the chordae tendineae 16 result in leaflet flail and prolapse, respectively. Leaflet flail and prolapse can also occur due to the chordae tendineae 16 stretching out. Leaflet flail, prolapse, rigidity, and/or other leaflet issues can result in a failure of coaptation, resulting in regurgitant blood flow.

FIG. 3B shows a mitral valve 6 where the annulus is dilated and deformed causing mitral regurgitation through the commissure 21. At a structural level, four general types of structural changes of the mitral valve apparatus can cause regurgitation: leaflet retraction from fibrosis and calcification, annular dilation, chordal abnormalities (including rupture, elongation, shortening, or apical tethering or “tenting” as seen in FMR and IMR), and possibly papillary muscle dysfunction.

Throughout the document, description and drawings often refer to the left chambers, and specifically to the mitral valve 6 and coronary sinus, as examples for the various implementations described. It is to be noted, however, that the various implementations and applications described can be utilized on other valves (e.g., tricuspid valve, pulmonary valve, aortic valve, etc.) and other vasculature (e.g., coronary artery, etc.) mutatis mutandis, as can be appreciated by those skilled in the art.

Several implementations and applications herein are directed towards systems, apparatuses, devices, etc. (e.g., leaflet repair systems, arrestor systems, prolapse repair systems, flail repair systems, repair systems, treatment systems, etc.) that arrest or otherwise treat valve leaflet issues, such as flail, prolapse, rigidity, etc. In some implementations, a system, apparatus, device, etc. herein is capable of being situated at the influent side of a valve such that it can apply contact pressure or support onto a region of flail, prolapse, rigidity, etc. The contact pressure or support provided by various implementations can help tighten, flatten out, and/or reshape the flail, prolapse, rigidity, and/or abnormality, which helps to extend the coapting edge of a leaflet back towards the coaptation area when in a closed position. Proper coaptation that results in a fully closed valve prevents valve regurgitation. In some implementations, the system, apparatus, device, etc. is configured to support, arrest, and/or depress a leaflet to prevent the leaflet from flailing or flipping towards the influent side of the valve. Likewise, in some implementations, the system, apparatus, device, etc. is configured to support, arrest, and/or depress a leaflet to prevent the leaflet from prolapsing or from protruding or bulging towards the influent side of the valve.

In some implementations, a system, apparatus, device, etc. herein (e.g., leaflet repair system, arrestor system, prolapse repair system, flail repair system, repair system, etc.) includes (but is not limited to) one face that is to directly contact the face of a leaflet experiencing leaflet issues, e.g., flail, prolapse, rigidity, etc. Typically, the influent face of a leaflet is the face that experiences flail, prolapse, rigidity, and/or other issues. In some implementations, the contact face of the device is contoured to the influent face of a leaflet, which can be a hyperbolic paraboloid-like contour. In some implementations, the contact face of the system/device provides contact pressure on a leaflet flail, prolapse, rigidity, and/or abnormality. In some implementations, the contact face has a width and a length such that it can cover the region of the leaflet experiencing flail, prolapse, rigidity, and/or abnormality. In some implementations, the length of the system/device extends into the coaptation area of the leaflet. In some implementations, the coaptation portion of the system/device helps promote coaptation of the leaflets when closed.

In some implementations, a system and/or device herein (e.g., leaflet repair system/device, arrestor system/device, prolapse repair system/device, flail repair system/device, repair system/device, treatment systems/devices, etc.) comprises wire form frame and/or a wire form device (e.g., a device comprising a wire form frame). Any appropriate material to produce a wire form can be utilized, including (but not limited to) nitinol, cobalt-chrome (CoCr), stainless steel, titanium, polyglycolic acid (PGA), polylactic acid (PLA), poly-D-lactide (PDLA), polyurethane (PU), poly-4-hydroxybutyrate (P4HB), polycaprolactone (PCL), polyether ether ketone (PEEK), cyclic olefin copolymers (COCs), poly ethylene vinyl acetate (EVA), polytetrafluorethylene (PTFE), perfluoroether (PFA), fluorinated ethylene propylene (FEP), additives thereof, and derivatives thereof. In some implementations, a wire form device or wire form frame is contractible, which is useful to fit within a catheter in a more compact or collapsed configuration for less invasive catheter delivery methodologies. In some implementations, nitinol is utilized for its self-expanding properties, which can be useful to implant the device in less invasive catheter delivery methodologies.

Various shapes of wire form devices or wire form frames can be utilized in various different implementations and applications. In some implementations, a wire form frame/device is shaped to have portions of the wire form provide contact pressure or support on the valve or leaflet issue, e.g., on the flail, prolapse, rigidity of a leaflet, misshapen valve portion, etc.

In some implementations, a wire form frame/device has length and width to surround an area of flail or prolapse and utilizes a sheet extending across the area to provide contact pressure on the flail, prolapse, rigidity, etc.

In some implementations, a wire form frame or wire form device has length and width to surround an area of flail or prolapse and utilizes wire that undulates or intersects across the area to provide contact pressure on the flail, prolapse, rigidity, etc.

In some implementations, a wire form frame or wire frame device is free of wire at an internal portion of the coaptation area devoid of wire such that any future procedures that may be needed at some later time can still be performed on the native leaflet coaptation area (e.g., edge to edge repair, such as suturing or clamping leaflet edges together).

In some implementations, a wire form frame or wire form device includes a support or counterforce support extending from the portion of the wire form device opposite of the coaptation area, which can help the wire form device provide contact pressure on the valve (e.g., on an area of flail, prolapse, rigidity, etc.). In some implementations, the support or counterforce support is configured to contact a heart chamber wall (e.g., atrium or ventricle wall). In some implementations, a wire form device includes an indentation or hook formed via the wire, which can help secure the device within the site of implantation by fitting within or hooking onto the commissures, clefts, or other similar valve areas.

In some implementations, a system and/or device herein (e.g., leaflet repair system/device, arrestor system/device, prolapse repair system/device, flail repair system/device, repair system/device, valve repair system/device, etc.) incorporates a sheet attached on a wire form capable of forming a contact face. In some implementations, a sheet provides a surface capable of providing contact pressure or support onto a portion of a valve or leaflet experiencing issues, such as flail, prolapse, rigidity, undesirable shape, etc.

Sheets herein can be impermeable, semi-permeable, or permeable to fluids (e.g., blood or plasma). In some implementations, the sheet is a mesh. In some implementations, a mesh can be formed utilizing interleaving strings that overlap and intersect. In some implementations, a mesh or permeable sheet can beneficially provide contact pressure/support without restricting the flow of blood or plasma, which can be important in various implementations. For instance, an impermeable sheet may trap blood or plasma between the device and leaflet, which in turn might create undesired pressures with the valve and/or create pressures that dislodges the device or alters its position.

In some implementations, the sheet is partially an impermeable material and partially a permeable mesh. For instance, in some implementations, a cooptation portion of a system/device herein utilizes an impermeable material while a non-coaptation portion of the device utilizes a permeable mesh. In some implementations, the impermeable coaptation portion helps promote proper closure of a native valve when coapting. In some implementations, a mesh is formed utilizing a mesh sheet.

Any appropriate material can be utilized for a sheet and/or mesh herein, including (but not limited to) poly(lactic-co-glycolic) acid (PLGA), polyvinylchloride (PVC), polyethylene (PE), polypropylene (PP), polytetrafluoroethylene (PTFE), polyurethane (PU), polyethylene terephthalate (PET), polyethersulfone (PES), polyglycolic acid (PGA), polylactic acid (PLA), poly-D-lactide (PDLA), poly-4-hydroxybutyrate (P4HB), and polycaprolactone (PCL).

Any appropriate means to attach a sheet and/or mesh onto a wire form can be utilized, including (but not limited to) stitching, staples, and glue. Optionally, in some implementations, the sheet is a form-fitted cover that stretches across the wire form or wire form frame.

In some implementations, a wire form device or a system/device having a wire form frame has a static portion and a dynamic portion. In some implementations, the static portion is capable of situating within the valve and can include indents and or hooks to secure the device within the site of implantation by fitting with or hooking onto the commissures or other similar leaflet areas. In some implementations, the dynamic portion includes a sheet to help provide contact pressure on and/or support to a leaflet, e.g., to address flail, prolapse, rigidity, etc. In some implementations, the dynamic portion is capable of being repositioned and/or resized during the implantation process such that it can be adequately cover the leaflet region experiencing the flail, prolapse, rigidity, and/or other issue.

Various implementations and applications of devices herein are to be used on any leaflet experiencing flail or prolapse. Accordingly, in some implementations, a device is capable of being utilized on a leaflet of a mitral, a tricuspid, an aortic, and/or a pulmonic valve. Likewise, various implementations and applications of devices can be utilized on any area of the leaflet experiencing flail or prolapse. In some implementations, a device is capable of being utilized on or near a leaflet commissure and/or any area between a leaflet's commissures.

To reach the site of implantation, any appropriate surgical, minimally invasive, or percutaneous technique can be utilized, including (but not limited to) a transcatheter delivery system, which can utilize a transfemoral, subclavian, transapical, transseptal, or transaortic approach. In some implementations, a delivery catheter is utilized to incorporate a device, then delivered to the site of deployment via a guidewire and utilized to anchor the device at the site of implantation.

Some implementations are directed to methods of delivering a device to the site of deployment. The various techniques, methods, operations, steps, etc. described or suggested anywhere herein (including in documents incorporated by reference herein) can be performed on a living animal (e.g., human, mammal, other animal, etc.) or on a non-living simulation, such as on a cadaver, cadaver heart, simulator (e.g., with the body parts, tissue, etc. being simulated), etc. Accordingly, methods of delivery include both methods of treatment (e.g., treatment of human subjects) and methods of training and/or practice (e.g., utilizing an anthropomorphic phantom that mimics human vasculature to perform method).

### Valve Treatment Devices and Procedures

Eliminating and/or tightening excess tissue at a leaflet may be one method for correcting valve issues, such as leaflet flail and/or prolapse. Surgical plication is a method in which a physician can eliminate excess leaflet tissue by creating a fold and/or tensioning the leaflet using suture stitching. However, such methods may require surgery and/or may be highly invasive. Some example solutions for leaflet flail and/or prolapse described herein can be configured for use on a beating and/or may involve transcatheter and/or percutaneous delivery of one or more implants.

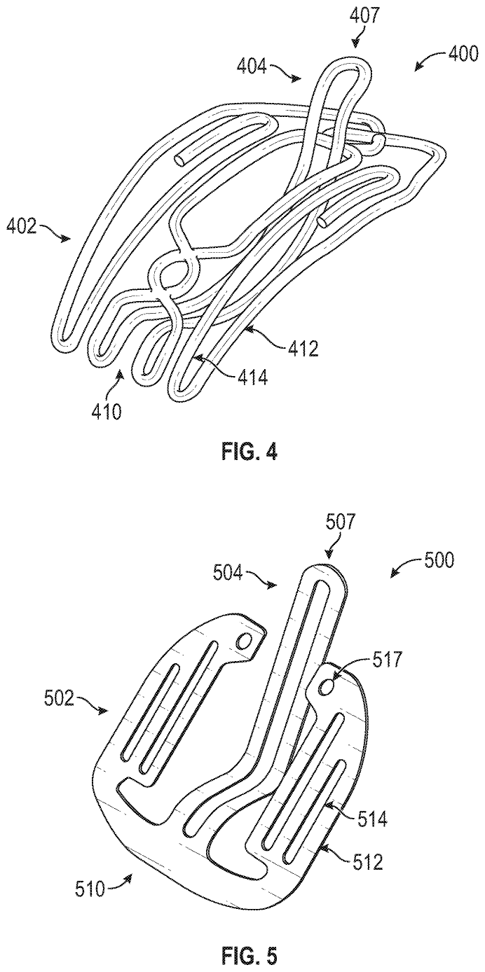

FIG. 4 illustrates an example implant 400 configured for attachment to one or more leaflets in accordance with one or more examples. In some implementations, the implant 400 can be configured to support and/or shape (e.g., reshape) one or more leaflets of a heart valve. For example, the implant 400 can be configured to attach to a leaflet to move the leaflet towards a plane of the heart valve and/or to prevent excessive flail of the leaflet.

The implant 400 can comprise a generally flat and/or laser-cut device. The implant 400 can be at least partially composed of one or more shape-memory alloys (e.g., nitinol) and/or can comprise a wire-form. In some implementations, the implant 400 can comprise a clip and/or can be configured to clip onto a free end of a leaflet. A physician can create a fold by displacing tissue and/or pinching/clipping the leaflet with the implant 400.

In some implementations, the implant 400 can comprise a base 402 and/or an arm 404. The arm 404 can be configured to move at least partially independently of the base 402. For example, the arm 404 can comprise a free end 407 and/or can be configured to form a single attachment point 410 to the base 402. The arm 404 can extend from the base 402 and/or can be configured to attach to the base 402.

The implant 400 can comprise a network of wires 412 (e.g., struts, cords, lines, etc.) extending in various directions and/or interconnecting. In some implementations, the wires 412 can form cells 414 between the struts 412. The arm 404 can be configured to pass at least partially through one or more cells 414 of the base 402 in a closed and/or default form of the implant 400. For example, the arm 404 can have a generally curved form and/or can be configured to curve away from the base 402 at or near the attachment point 410 and/or to curve towards the base 402 at or near the free end 407 of the arm 404.

The arm 404 can comprise an at least partially curved wire. In some implementations, the arm 404 can comprise a loop and/or looped portion of the wires 412. For example, the arm 404 can comprise two lines of wire 412 extending generally in parallel and/or joining in a curve (e.g., semi-circle) at the free end 407 of the arm 404.

The base 402 can have a generally curved form. For example, the base 402 can have a generally convex and/or concave form and/or can be configured to approximate a curvature of one or more leaflets. In some implementations, the arm 404 can have a generally opposite curvature to the base 402. For example, between the attachment point 410 and the free end 407, the base 402 and the arm 404 can extend outwardly in generally opposite directions.

The implant 400 can have a generally elastic form and/or can be configured to naturally move towards the default form shown in FIG. 4. During a delivery process, the arm 404 can be configured to be pulled away from the base 402 to create a greater separation between the free end 407 and the base 402 to allow one or more leaflets to slide between the arm 404 and the base 402.

The implant 400 can comprise a covering and/or sheet extending at least partially over the implant 400. For example, a sheet can extend at least partially across the base 402 and/or along the arm 404 to extend a surface area of the implant 400.

FIG. 5 illustrates another example implant 500 configured for attachment to one or more leaflets in accordance with one or more examples. In some implementations, the implant 500 can be configured to support and/or shape one or more leaflets of a heart valve. For example, the implant 500 can be configured to attach to a leaflet to move the leaflet towards a plane of the heart valve and/or to prevent excessive flail of the leaflet.

The implant 500 can comprise a generally flat and/or laser-cut device. The implant 500 can be at least partially composed of one or more shape-memory alloys (e.g., nitinol) and/or can comprise a wire-form. In some implementations, the implant 500 can comprise a clip and/or can be configured to clip onto a free end of a leaflet. A physician can create a fold by displacing tissue and/or pinching/clipping the leaflet with the implant 500.

In some implementations, the implant 500 can comprise a base 502 and/or an arm 504. The arm 504 can be configured to move at least partially independently of the base 502. For example, the arm 504 can comprise a free end 507 and/or can be configured to form a single attachment point 510 to the base 502. The arm 504 can extend from the base 502 and/or can be configured to attach to the base 502.

The implant 500 can comprise a network of wires 512 (e.g., struts, cords, lines, etc.) extending in various directions and/or interconnecting. In some implementations, the wires 512 can form cells 514 between the struts 512. The arm 504 can be configured to pass at least partially between struts 512 and/or cells 514 of the base 502 in a closed and/or default form of the implant 500. For example, the arm 504 can have a generally curved form and/or can be configured to curve away from the base 502 at or near the attachment point 510 and/or to curve towards the base 502 at or near the free end 507 of the arm 504. The base 502 can extend generally in parallel with the arm 504 on either side of the arm 504 and/or can form a gap to accommodate the arm 504.

The arm 504 can comprise an at least partially curved wire 512 and/or strut. In some implementations, the arm 504 can comprise a loop and/or looped portion of the wires 512. For example, the arm 504 can comprise two lines of wire 512 extending generally in parallel and/or joining in a curve (e.g., semi-circle) at the free end 507 of the arm 504.

The base 502 can have a generally straight form. For example, the base 502 can extend generally along a first plane and/or the arm 504 can curve to an approximately 45-degree angle away from the plane and/or towards the plane.

The implant 500 can have a generally elastic form and/or can be configured to naturally move towards the default form shown in FIG. 5. During a delivery process, the arm 504 can be configured to be pulled away from the base 502 to create a greater separation between the free end 507 and the base 502 to allow one or more leaflets to slide between the arm 504 and the base 502.

In some implementations, the implant 500 can comprise one or more apertures 517 configured to facilitate attachment of one or more cords and/or sutures for stabilizing and/or causing movement of the base 502 and/or arm 504. The base 502 can comprise one or more apertures 517 at ends of the base 502 and/or the arm 504 can similarly comprise or more apertures 517 at or near a free end 507 of the arm 504.

The implant 500 can comprise a covering and/or sheet extending at least partially over the implant 500. For example, a sheet can extend at least partially across the base 502 and/or along the arm 504 to extend a surface area of the implant 500.

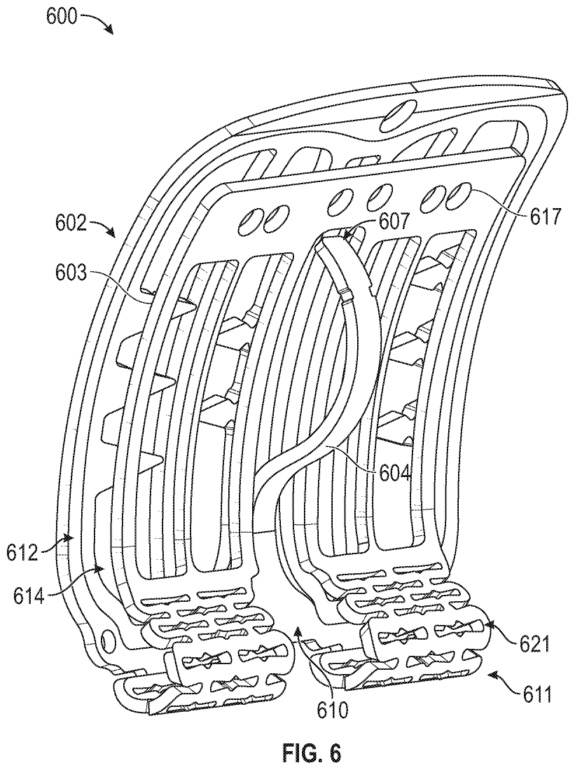

FIG. 6 illustrates another example implant 600 configured for attachment to one or more leaflets in accordance with one or more examples. In some implementations, the implant 600 can be configured to support and/or shape one or more leaflets of a heart valve. For example, the implant 600 can be configured to attach to a leaflet to move the leaflet towards a plane of the heart valve and/or to prevent excessive flail of the leaflet.

The implant 600 can comprise a generally flat and/or curved laser-cut device. The implant 600 can be at least partially composed of one or more shape-memory alloys (e.g., nitinol) and/or can comprise a wire-form. In some implementations, the implant 600 can comprise a clip and/or can be configured to clip onto a free end of a leaflet. A physician can create a fold by displacing tissue and/or pinching/clipping the leaflet with the implant 600.

In some implementations, the implant 600 can comprise a base 602, a clamp 603, and/or an arm 604. The arm 604 can be configured to move at least partially independently of the base 602. For example, the arm 604 can comprise a free end 607 and/or can be configured to form a single attachment point 610 to the base 602. The arm 604 can extend from the base 602 and/or can be configured to attach to the base 602.

The clamp 603 can have a generally flat and/or curved form and/or can approximate a shape and/or curvature of the base 602. In some implementations, the clamp 603 can have a smaller surface area and/or size than the base 602. For example, the clamp 603 can be configured to extend along a portion of the base 602 and/or may not have a sufficient size to extend along an entire surface area of the base 602. In some implementations, the clamp 603 can be coupled to the base 602 via one or more connectors 611. The connectors 611 can comprise a generally curved and/or flexible sheet of metal, plastic, and/or other suitable material. In some implementations, the connectors 611 can comprise one or more cavities 621 configured to facilitate bending and/or movement of the connectors.

In some implementations, the connectors 611 can be configured to form a hinged connection between the clamp 603 and the base 602. For example, the connectors 611 can connect to a first side of the clamp 603 and/or a first side of the base 602 while allowing the base 602 and/or clamp 603 to swing freely from the first side. In some implementations, the implant 600 can comprise two connectors 611 configured to couple to multiple points at the first sides of the clamp 603 and/or base 602.

The base 602 and the clamp 603 can be configured to be pulled apart and/or to move apart to allow one or more leaflets to fit between the base 602 and the clamp 603. Once the one or more leaflets are positioned between the base 602 and the clamp 603, the base 602 and the clamp 603 can be configured to close together to clamp the one or more leaflets in place. In some implementations, the base 602 and/or clamp 603 can be shape-set to a clamping position in which the base 602 and the clamp 603 are relatively close together. However, the base 602 and the clamp 603 can be configured to be elastically deformed due to a generally elastic structure of the connectors 611.

The implant 600 can comprise a network of wires 612 (e.g., struts, cords, lines, etc.) extending in various directions and/or interconnecting. In some implementations, the wires 612 can form cells 614 between the struts 612. The arm 604 can be configured to pass at least partially through one or more cells 614 of the base 602 and/or clamp 603 in a closed and/or default form of the implant 600. For example, the arm 604 can have a generally curved form and/or can be configured to curve away from the base 602 and/or clamp 603 at or near the attachment point 610 and/or to curve towards the base 602 and/or clamp 603 at or near the free end 607 of the arm 604.

The arm 604 can comprise an at least partially curved wire. In some implementations, the arm 604 can comprise a single line and/or a loop and/or looped portion of the wires 612. For example, the arm 604 can comprise a looped line of wire 612 extending generally in parallel and/or joining in a curve (e.g., semi-circle) at the free end 607 of the arm 604.

The base 602 and/or clamp 603 can have a generally curved form. For example, the base 602 and/or clamp 603 can have a generally convex and/or concave form and/or can be configured to approximate a curvature of one or more leaflets. In some implementations, the arm 604 can have a generally opposite curvature to the base 602 and/or clamp 603. For example, between the attachment point 610 and the free end 607, the base 602 and the arm 604 can extend outwardly in generally opposite directions.

The implant 600 can have a generally elastic form and/or can be configured to naturally move towards the default form shown in FIG. 6. During a delivery process, the arm 604 can be configured to be pulled away from the base 602 and/or clamp 603 to create a greater separation between the free end 607 and the base 602 and/or clamp 603 to allow one or more leaflets to slide between the arm 604 and the base 602 and/or clamp 603.

In some implementations, the implant 600 can comprise one or more apertures 617 configured to facilitate attachment of one or more cords and/or sutures for stabilizing and/or causing movement of the base 602, clamp 603, and/or arm 604. The base 602 and/or clamp 603 can comprise one or more apertures 617 at ends of the base 602 and/or clamp 603 and/or the arm 604 can similarly comprise or more apertures 617 at or near a free end 607 of the arm 604.

The implant 600 can comprise a covering and/or sheet extending at least partially over the implant 600. For example, a sheet can extend at least partially across the base 602 and/or along the arm 604 to extend a surface area of the implant 600.

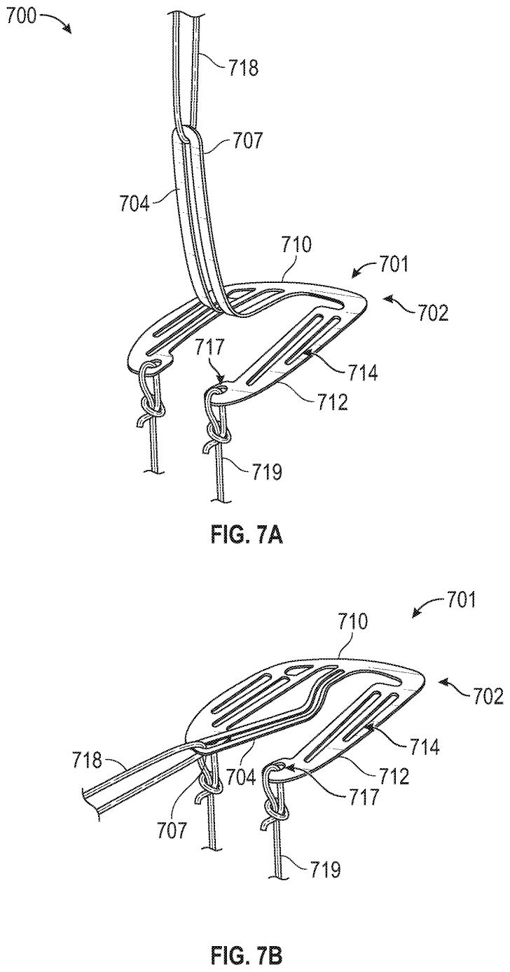

FIGS. 7A and 7B illustrate movement of an example repair system 700 comprising an example implant 701 in accordance with one or more examples. FIG. 7A illustrates the implant 701 in an open form and FIG. 7B illustrates the implant 701 in a closed form. The implant 701 can be configured to support and/or shape one or more leaflets of a heart valve. For example, the implant 701 can be configured to attach to a leaflet to move the leaflet towards a plane of the heart valve and/or to prevent excessive flail of the leaflet.

The implant 701 can comprise a generally flat and/or laser-cut device. The implant 701 can be at least partially composed of one or more shape-memory alloys (e.g., nitinol) and/or can comprise a wire-form. In some implementations, the implant 701 can comprise a clip and/or can be configured to clip onto a free end of a leaflet. A physician can create a fold by displacing tissue and/or pinching/clipping the leaflet with the implant 701.

In some implementations, the implant 701 can comprise a base 702 and/or an arm 704. The arm 704 can be configured to move at least partially independently of the base 702. For example, the arm 704 can comprise a free end 707 and/or can be configured to form a single attachment point 710 to the base 702. The arm 704 can extend from the base 702 and/or can be configured to attach to the base 702.

FIGS. 7A and 7B illustrate use of a first cord 718 (e.g., suture) to cause movement and/or stabilization of the arm 704 and/or one or more second cords 719 to cause movement and/or stabilization of the base 702. For example, the first cord 718 can be used to pull the arm 704 away from the base to increase a separation between the arm 704 and the base 702 to allow for insertion of one or more leaflets and/or folds of leaflets between the arm 704 and the base 702. In some implementations, the one or more second cords 719 can additionally or alternatively be used to pull the base 702 away from the arm 704. However, the first cord 718 and/or the second cords 719 can be used to stabilize and/or hold the arm 704 and/or base 702 in place. The cords 718, 719 are shown for illustrative purposes. However, cords 718, 719 may not be required to facilitate delivery of one or more leaflets between the base 702 and the arm 704 and/or only the first cord 718 can be used. The cords 718, 719 can be configured to pass through apertures 717 of the implant 701.

The implant 701 can comprise a network of wires 712 (e.g., struts, cords, lines, etc.) extending in various directions and/or interconnecting. In some implementations, the wires 712 can form cells 714 between the struts 712. The arm 704 can be configured to pass at least partially between struts 712 and/or cells 714 of the base 702 in a closed and/or default form of the implant 701. For example, the arm 704 can have a generally curved form and/or can be configured to curve away from the base 702 at or near the attachment point 710 and/or to curve towards the base 702 at or near the free end 707 of the arm 704. The base 702 can extend generally in parallel with the arm 704 on either side of the arm 704 and/or can form a gap to accommodate the arm 704.

The arm 704 can comprise an at least partially curved wire 712 and/or strut. In some implementations, the arm 704 can comprise a loop and/or looped portion of the wires 712. For example, the arm 704 can comprise two lines of wire 712 extending generally in parallel and/or joining in a curve (e.g., semi-circle) at the free end 707 of the arm 704.

The base 702 can have a generally straight form. For example, the base 702 can extend generally along a first plane and/or the arm 704 can curve to an approximately 45-degree angle away from the plane and/or towards the plane.

The implant 701 can have a generally elastic form and/or can be configured to naturally move towards the default form shown in FIG. 7. During a delivery process, the arm 704 can be configured to be pulled away from the base 702 to create a greater separation between the free end 707 and the base 702 to allow one or more leaflets to slide between the arm 704 and the base 702.

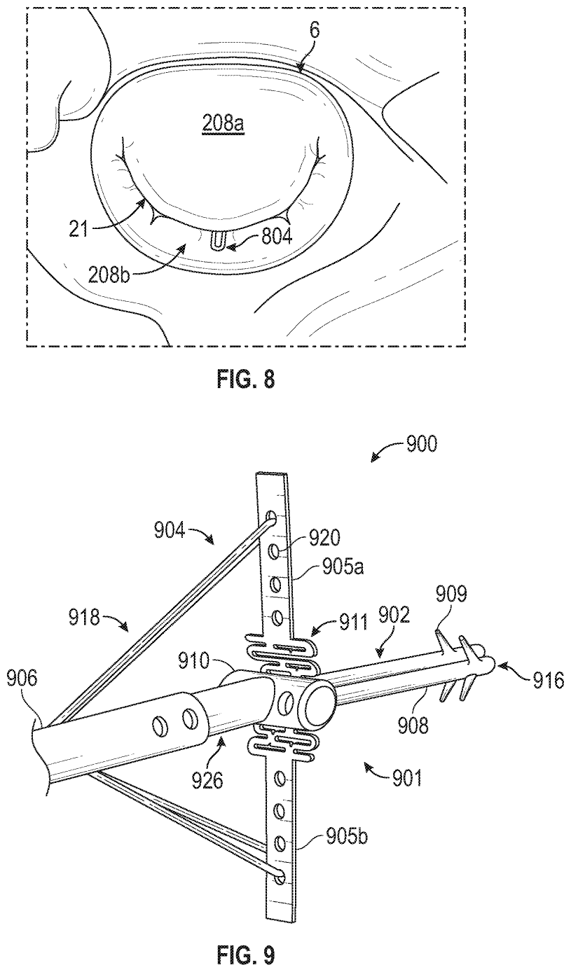

FIG. 8 illustrates an example implant anchored and/or clipped onto a posterior leaflet 208b of a mitral valve 6 in accordance with one or more examples. The implant can comprise an arm 804 configured to extend through a commissure 21 of the valve 6 and/or onto an upper surface of the posterior leaflet 208b. The implant can comprise a base (not shown) clipped onto and/or anchored to a lower surface of the posterior leaflet 208b.

The implant can be configured to be delivered via a catheter and/or can be advanced to a heart chamber that is adjacent to (e.g., upstream of) the heart valve 6 that is to be treated. The catheter can be advanced to the chamber prior to advancing the implant through the catheter, or the catheter can be advanced to the chamber with the implant already disposed therein. In the example of treating a mitral valve 6 of a heart, the implant can be advanced to left atrium of the heart. The mitral valve 6 has a first leaflet (e.g., a posterior leaflet 208b) and an opposing leaflet (e.g., an anterior leaflet 208a). In the illustrated example, the posterior leaflet 208b is the leaflet that is experiencing flail. It is to be noted that implants can similarly be used to treat flail in anterior leaflet 208a, mutatis mutandis.

The catheter can be advanced to the heart chamber transluminally. However, a transatrial approach is also within the scope of the disclosure. Similarly, although a transfemoral approach is shown, the scope of the disclosure includes advancement via the superior vena cava. Advancement of implant within catheter can be performed while a shaft (e.g., head thereof) is engaged with an interface (e.g., proximal end) of the implant.

Optimality of a given position of implant can be determined during the implantation procedure, e.g., prior to anchoring the implant to the tissue. For example, optimality can be determined using blood pressure sensing and/or imaging techniques such as fluoroscopy and echocardiography. For example, Doppler echocardiography can be used to determine a degree to which regurgitation through the valve remains or has been reduced.

FIG. 9 illustrates an example treatment and/or repair system 900 configured to percutaneously and/or transcatheter treat, repair, and/or assist a valve (e.g., one or more leaflets of a valve). The system 900 can comprise a device or implant 901 configured for delivery via a catheter 906 and/or other delivery device.

The device or implant 901 can comprise a clamping portion 902 and/or a locking portion 904. The clamping portion 902 can comprise two or more elongate fingers 908 configured to extend generally in parallel in a closed position of the clamping portion 902. The fingers 908 can form a hinge and/or similar connection to the locking portion 904. In some implementations, the fingers 908 can be configured to be pulled apart to increase a mouth and/or slot between the fingers 908 and/or to be moved together to fully and/or nearly close the mouth and/or slot between the fingers 908. For example, the fingers 908 can be pulled apart to allow one or more leaflets to slide between the fingers 908. With the one or more leaflets disposed between the fingers 908, the fingers 908 can be closed to clamp the one or more leaflets.

While described by way of example as an implant in various examples, the device 901 need not be implanted and remain in the body, e.g., the same or similar concepts to those described herein could be used to reshape a portion of the valve and/or leaflet and then be removed, be used to temporarily reshape a portion of the valve and/or leaflet while another repair and/or implantation step using other devices is done, be used in combination with other treatments and/or repairs, be use to clamp and/or reshape and then apply energy and or adhesive to treat and/or repair the valve and/or leaflet, etc.

The fingers 908 can comprise various attachment and/or anchoring elements configured to facilitate attachment and/or anchoring to one or more leaflets and/or surrounding tissue. In some implementations, one or more of the fingers 908 can comprise barbs 909 (e.g., protrusions, pins, needles, etc.) extending outwardly and/or inwardly from the fingers 908. The barbs 909 can be disposed at or near distal ends 916 of the fingers 908. In some implementations, one or more fingers 908 can comprise multiple barbs 909. For example, one or more fingers 908 can comprise two barbs disposed at the distal ends 916 of the fingers 908. The barbs 909 can extend generally diagonally (e.g., at an approximately 45-degree angle) relative to the fingers 908 and/or can be pointed and/or angled towards the locking portion 904. In some implementations, the barbs 909 can be configured to improve retention of the implant 901 with the surrounding leaflets.

The device or implant 901 can comprise a locking portion 904 configured to lock the one or more fingers 908 and/or folds of one or more leaflets at or near the implant 901. The locking portion 904 can comprise one or more paddles 905 (e.g., arms, elongate members, locking members, etc.) configured to move relative to and/or independently of the one or more fingers 908. The paddles 905 and/or fingers 908 can have hinged attachments to a base portion 910 of the implant 901.

While the paddles 905 are shown at an approximately 90-degree angle relative to the fingers 908 and/or base portion 910, the paddles 905 can be configured to move to any angle relative to the fingers 908 and/or base portion 910. For example, the paddles 905 can be configured to lay flatly against and/or adjacent to the fingers (e.g., at an approximately 0-degree angle relative to the fingers 908) and/or to lay flatly against the delivery device 906 and/or base portion 910 (e.g., at an approximately 180-degree angle relative to the fingers 908).

In some implementations, the paddles 905 can be biased towards a 0-degree angle relative to the fingers 908. For example, in the absence of external forces, the paddles 905 may naturally lay flatly against the fingers 908 and/or extend generally in parallel to each other. In some implementations, the paddles 905 can be pulled and/or held in a first and/or locked position during delivery into the body and/or to a target location. The system 900 can comprise one or more wires 918 (e.g., arms, lines, tendons, etc.) configured to pull and/or hold the paddles 905 away from the default form. For example, as shown in FIG. 9, the one or more wires 918 can be configured to hold the paddles 905 at a generally 90-degree angle relative to the fingers 908. However, the one or more wires 918 can be configured to hold the paddles 905 at a generally 180-degree angle relative to the fingers 908. The system 900 can comprise any number of wires 918. For example, each paddle 905 can be coupled to and/or held by one or more wires 918.

The paddles 905 can have rectangular and/or generally flat shapes. However, other shapes and/or sizes can be suitable. In some implementations, the paddles 905 can comprise one or more apertures 920 (e.g., openings, holes, etc.). The apertures 920 can be configured to receive the wires 918 to facilitate coupling of the wires 918 to the paddles 905. The paddles 905 can comprise four apertures 920 but can comprise any suitable number of apertures 920.

The locking portion 904 can comprise multiple paddles 905. For example, the locking portion 904 can comprise a first paddle 905a and/or a second paddle 905b. The first paddle 905a and the second paddle 905b can be disposed generally opposite each other across the base 910. In some implementations, each of the paddles can be generally in-line with one of the fingers 908 and/or can be at least partially offset from the one or more fingers 908. In some implementations, the one or more paddles 905 can be configured to connect to the base 910 via one or more joints 911.

In some implementations, a joint 911 can comprise a network of wires and/or struts extending between the base 910 and one or more paddles 905. In some implementations, a joint 911 can have a generally bendable and/or flexible structure and/or can be configured to facilitate a movable and/or hinged attachment between the base 910 and the one or more paddles 905. For example, a joint 911 can be configured to allow a paddle 905 to swing between a 0-degree angle (e.g., laying approximately flushly with the fingers 908) and a 180-degree angle (e.g., laying approximately flushly against the catheter 906 and/or a proximal end 926 of the implant 901.

In some implementations, the one or more paddles 905 can be configured to lock and/or hold one or more folds of a leaflet and/or other tissue. For example, the one or more paddles 905 can be configured to naturally and/or elastically move towards the fingers 908 to clamp one or more leaflets and/or folds of leaflets between the paddles 905 and the fingers 908.

The fingers 908 can be configured to create an opening and/or slot between the fingers 908 to allow one or more leaflets to pass between the fingers 908. In response to twisting of the catheter 906 and/or proximal end 926, the fingers 908 can be configured to twist and/or the barbs 909 can be configured to press into the leaflet to facilitate a secure grasp of the leaflet and/or to improve retention of the implant 901.

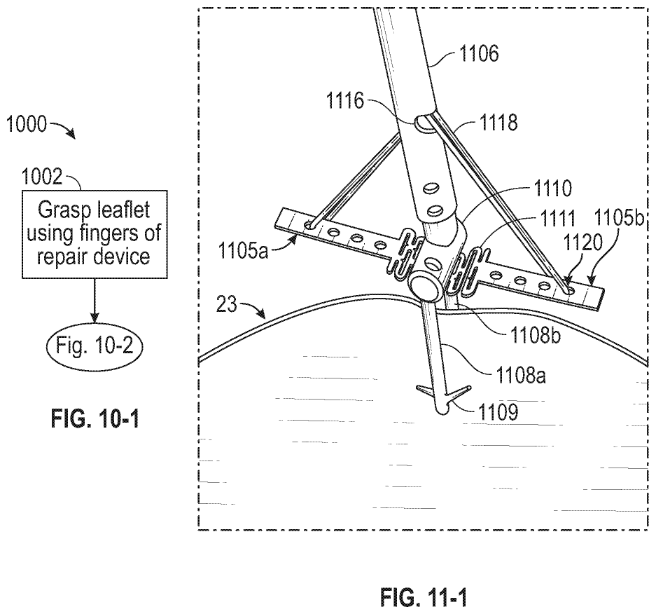

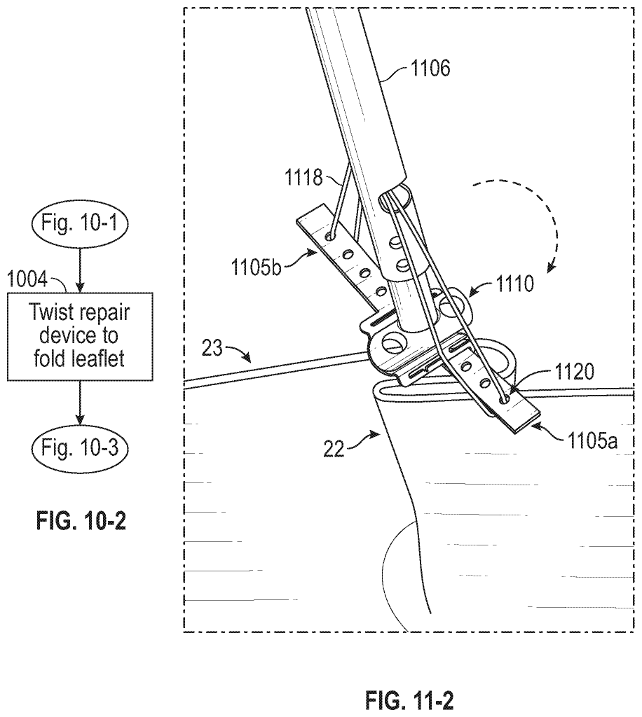

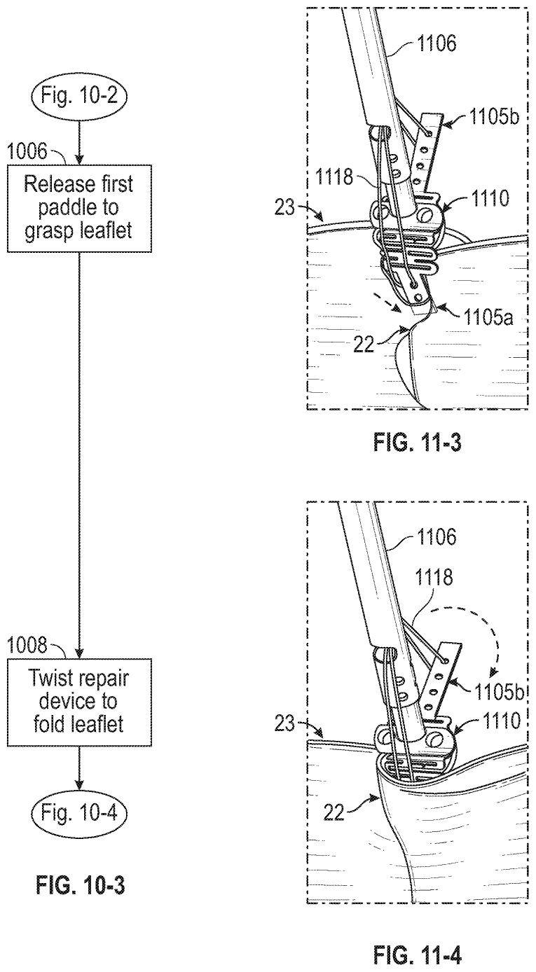

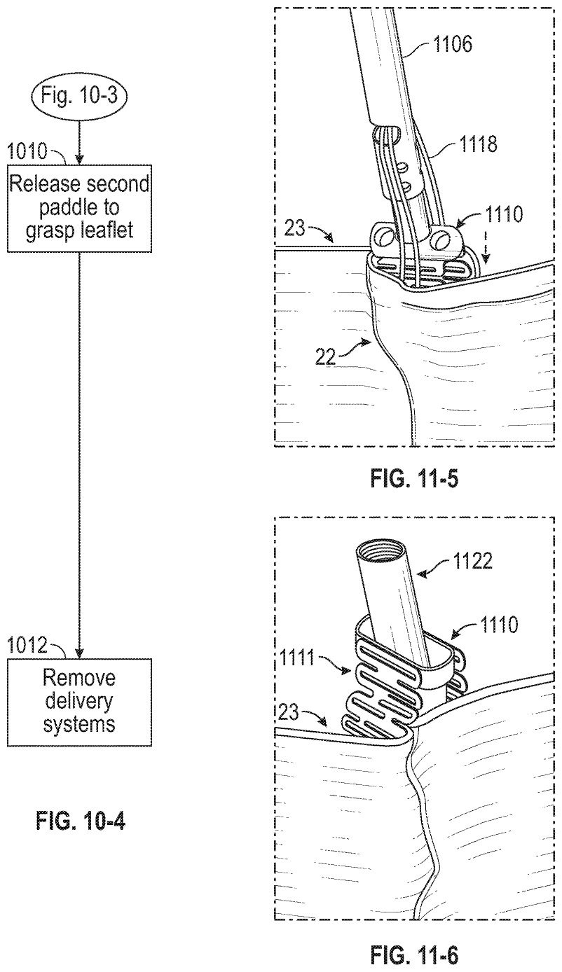

FIG. 10 (FIGS. 10-1, 10-2, 10-3, and 10-4) is a flowchart illustrating steps of an example method or process 1000 for delivering one or more implants in accordance with the present disclosure and/or grasping and/or treating one or more leaflets of a heart valve of a subject (e.g., of a living subject, of a simulation, etc.) using the devices or implants herein. FIG. 11 (FIGS. 11-1, 11-2, 11-3, 11-4, 11-5, and 11-6) provides certain images depicting various features associated with steps of the process 1000 depicted in FIG. 10.

At step 1002, the process 1000 involves grasping a leaflet 23 of a heart valve using one or more fingers 1108 of an implant and/or device, as illustrated in FIG. 11-1. In some implementations, the device/implant can comprise a first finger 1108a and/or a second finger 1108b with at least partial gap separating the first finger 1108a and the second finger 1108b. The first finger 1108a can be at least partially movable and/or can have a hinged attachment to a base portion 1110 of the implant. The gap between the first finger 1108a and the second finger 1108b can be sufficiently large to allow the leaflet 23 to slide between the first finger 1108a and the second finger 1108b.

In some implementations, the device/implant can be coupled to a delivery device 1106 (e.g., a catheter) when the implant grasps the leaflet 23. A physician can be enabled to guide the delivery device 1106 and/or implant to the leaflet 23 prior to removal of the implant from the delivery device 1106.

The one or more fingers 1108 can comprise various features configured to facilitate a grasp and/or attachment between the one or more fingers 1108 and the leaflet 23. For example, one or more fingers 1108 can comprise barbs 1109 and/or other features configured to extend outwardly from the one or more fingers 1108 and/or to press into the leaflet 23. In some implementations, the one or more barbs 1109 can be disposed at distal ends of the fingers 1108.

At step 1004, the process 1000 involves twisting the delivery device 1106 and/or implant, as shown in FIG. 11-2. In some implementations, twisting of the device/implant can be configured to cause folding of the leaflet 23 and/or the leaflet 23 can be pulled into one or more folds 22 in response to twisting of the implant.

In some implementations, the barbs 1109 of the device/implant can be configured to facilitate pulling of the leaflet 23 in response to twisting of the device/implant. Folding of the leaflet 23 can result in the leaflet 23 becoming more taught and/or tight. In some implementations, the device/implant can be twisted in a full rotation (e.g., approximately 360-degrees) and/or less than a full rotation.

In some implementations, the implant can comprise one or more paddles 1105 configured to be held away from the leaflet 23 during twisting of the implant. The paddles 1105 can be configured to connect to the base portion 1110 via one or more connectors 1111, which can form a hinged attachment between the base portion 1110 and the paddles 1105.

At step 1006, the process 1000 involves releasing a first paddle 1105a to allow the first paddle 1105a to move towards and/or into contact with the leaflet 23 and/or one or more folds 22 of the leaflet 23, as shown in FIG. 11-3. In some implementations, one or more wires 1118 (e.g., cords, sutures, etc.) attached to the first paddle 1105a can be released and/or loosened to allow the first paddle 1105a to move towards a naturally compressed state and/or towards the one or more fingers 1108 and/or the leaflet 23. The first paddle 1105a can be released when the first paddle 1105a is positioned over and/or adjacent to a fold 22 of the leaflet 23. The device/implant can stop rotating when the first paddle 1105a is released. The one or more wires 1118 can be configured to extend through one or more openings 1116 through the delivery device 1106. The one or more paddles 1105 can comprise cavities 1120 configured to receive the one or more wires 1118.

At step 1008, in some implementations, the process 1000 involves twisting the implant to create additional folds 22 in the leaflet 23, as shown in FIG. 11-4. In some implementations, twisting of the implant can cause the first paddle 1105 to be wrapped in the leaflet 23. The implant can be twisted in a full rotation (e.g., approximately 360-degrees) and/or less than a full rotation.

At step 1010, the process 1000 involves releasing a second paddle 1105b to allow the second paddle 1105b to move towards and/or into contact with the leaflet 23 and/or one or more folds 22 of the leaflet 23, as shown in FIG. 11-5. In some implementations, one or more wires 1118 (e.g., cords, sutures, etc.) attached to the second paddle 1105b can be released and/or loosened to allow the second paddle 1105b to move towards a naturally compressed state and/or towards the one or more fingers 1108 and/or the leaflet 23. The second paddle 1105b can be released when the second paddle 1105b is positioned over and/or adjacent to a fold 22 of the leaflet 23. The device/implant can stop rotating when the second paddle 1105b is released.

At step 1012, the process 1000 involves removing the delivery device 1106 and/or other delivery systems, as shown in FIG. 11-6. In some implementations, the device/implant can comprise a proximal end 1122 configured to mate with the delivery device 1106. The proximal end 1122 can have a generally tubular form and/or can be configured to fit at least partially within the delivery device 1106. In some implementations, the proximal end 1122 can comprise threads and/or can be threaded to facilitate coupling with the delivery device 1106.

In some implementations, steps of the process 1000 can be performed in any order.

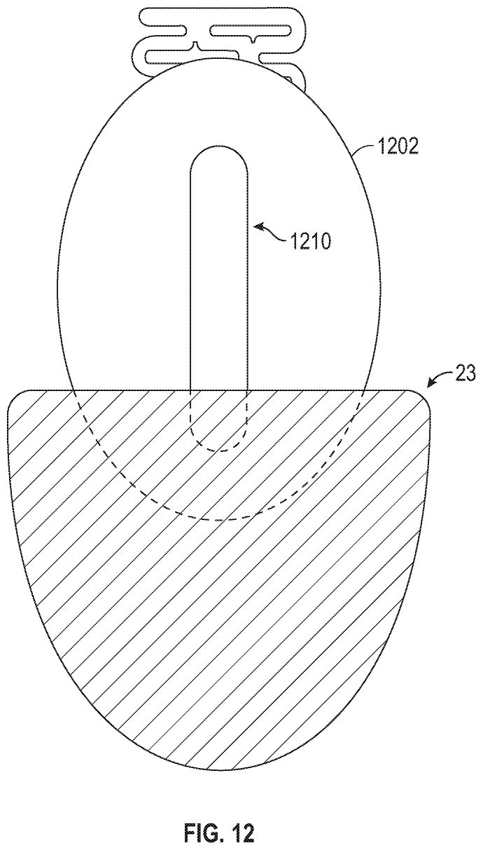

FIG. 12 illustrates a grasper 1202 of an example device or implant of the present disclosure. In some implementations, the grasper 1202 can be configured to grasp a leaflet 23 of a valve. The grasper 1202 can form a lumen 1210 and/or opening to allow the leaflet 23 to at least partially enter the lumen 1210. The grasper 1202 can be disposed at a distal end of an example implant.

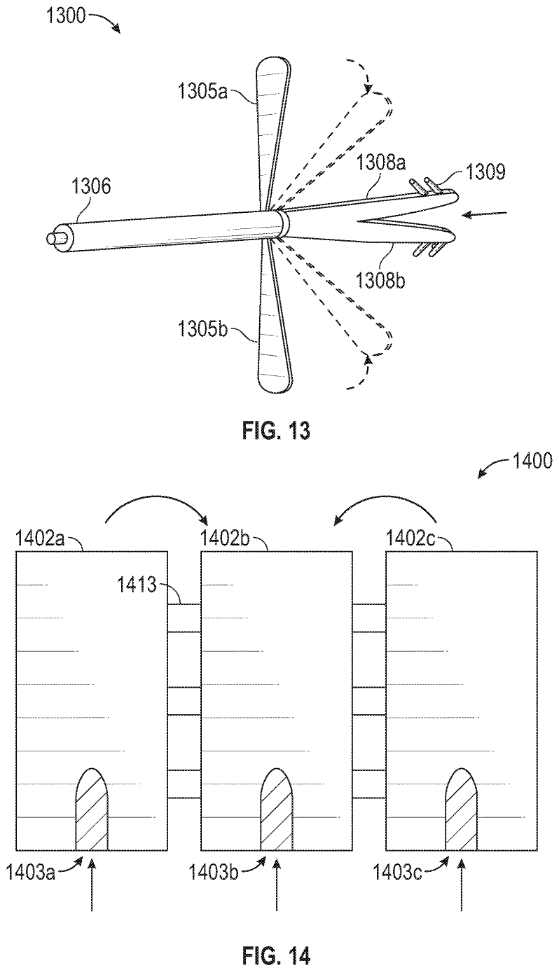

FIG. 13 illustrates an example device or implant 1300 in accordance with one or more examples. The device/implant 1300 can comprise one or more fingers 1308 configured to grasp one or more leaflets of a heart valve. In some implementations, the device/implant 1300 can comprise a first finger 1308a and/or a second finger 1308b configured to form an opening into which one or more leaflets can enter. The one or more fingers 1308 can comprise barbs 1309 and/or other features configured to facilitate grasping of the one or more leaflets (e.g., as the device/implant 1300 is twisted).

The device/implant 1300 can comprise one or more paddles 1305 configured to be movable to lock and/or press against one or more leaflets and/or folds of leaflets during a twisting and/or grasping process. In some implementations, the device/implant 1300 can comprise a first paddle 1305a and/or a second paddle 1305b. The one or more paddles 1305 can be configured to move towards the fingers 1308 to lock and/or grasp the one or more leaflets.

The device/implant 1300 can comprise a catheter 1306 and/or other delivery device. In some implementations, the catheter 1306 can be removable and/or can be detached from the device/implant 1300 following a delivery process.

FIG. 14 illustrates an example device or implant 1400 configured to grasp and/or clamp onto one or more leaflets of a heart valve. The device/implant 1400 can comprise one or more segments 1402 interconnected by connectors 1413.

In some implementations, the device/implant 1400 can comprise three segments, including a first segment 1402a, a second segment 1402b, and/or a third segment 1402. In some implementations, each of the segments 1402 can comprise a cavity 1403 configured to allow one or more leaflets to enter the cavity to facilitate grasping of the one or more leaflets. For example, the first segment 1402a can be configured to grasp one or more leaflets at a first cavity 1403a.

In some implementations, the one or more connectors 1413 can be configured to form hinged attachments between the segments 1402 such that the segments can be moved independently of each other and/or can be configured to move into contact with each other and/or to lay flatly against each other. For example, after the first cavity 1403a grasps the one or more leaflets, the second segment 1402b can be folded towards the first segment 1402a to clasp the one or more leaflets and/or the second cavity 1403b can be configured to grasp the one or more leaflets. Similarly, the third segment 1402c can be configured to fold onto the second segment 1402b and/or the third cavity 1403c can be configured to grasp the one or more leaflets.

In some implementations, the one or more segments 1402 are shown having a generally uniform and/or rectangular shape. However, the segments 1402 can have different sizes and/or shapes. In some implementations, one or more segments 1402 can have a triangular, circular, oval, and/or other shape. Each of the segments 1402 can have distinct and/or different sizes and/or shapes.

### Additional Features and Examples

Provided below is a list of examples, each of which may include aspects of any of the other examples disclosed herein. Furthermore, aspects of any example described above may be implemented in any of the numbered examples provided below.

Example 1: An implant for treating a heart valve comprising a base portion; and a first movable arm extending from the base portion and configured to move independently of the base portion to grasp a leaflet of the heart valve.

Example 2: The implant of any example herein, in particular example 1, wherein the base portion has a generally curved form.

Example 3: The implant of any example herein, in particular example 2, wherein the first movable arm has a generally curved form that is generally opposite a curvature of the base portion.

Example 4: The implant of any example herein, in particular example 1, wherein the base portion comprises a network of struts forming one or more cells and wherein the first movable arm is configured to extend through at least one of the one or more cells.

Example 5: The implant of any example herein, in particular example 1, wherein the base portion forms a gap between ends of the base portion and wherein the first movable arm is configured to extend between the ends of the base portion.

Example 6: The implant of any example herein, in particular example 1, wherein the first movable arm is configured to grasp the leaflet against the base portion.

Example 7: The implant of any example herein, in particular example 1, further comprising a clamp extending approximately in-line with the base portion and forming a gap from the base portion.

Example 8: The implant of any example herein, in particular example 1, further comprising a second movable arm extending from the base portion and configured to grasp the leaflet against the first movable arm.

Example 9: The implant of any example herein, in particular example 1, further comprising one or more paddles extending from the base portion and configured to naturally move towards the first movable arm.

Example 10: The implant of any example herein, in particular example 9, further comprising one or more wires configured to hold the one or more paddles away from the first movable arm.

Example 11: A method comprising grasping a leaflet of a heart valve with a first movable arm of an implant, the implant comprising a first paddle held distally from the first movable arm; twisting the implant to cause folding of the leaflet; and releasing the first paddle to cause the first paddle to grasp the leaflet against the first movable arm.

Example 12: The method of any example herein, in particular example 11, wherein the implant further comprises a second movable arm, the method further comprising grasping the leaflet between the first movable arm and the second movable arm.

Example 13: The method of any example herein, in particular example 11, wherein the first movable arm comprises one or more barbs configured to facilitate grasping of the leaflet.

Example 14: The method of any example herein, in particular example 11, wherein the implant further comprises a second paddle held distally from the first movable arm, the method further comprising releasing the second paddle to cause the second paddle to grasp the leaflet.

Example 15: The method of any example herein, in particular example 11, further comprising one or more cords coupled to the first paddle and configured to hold the first paddle distally from the first movable arm.

Example 16: The method of any example herein, in particular example 15, further comprising relaxing the one or more cords to release the first paddle.

Example 17: The method of any example herein, in particular example 15, wherein the one or more cords are configured to hold the first paddle at an approximately 90-degree angle relative to the first movable arm.

Example 18: The method of any example herein, in particular example 15, wherein the one or more cords are configured to hold the first paddle at an approximately 180-degree angle relative to the first movable arm.

Example 19: The method of any example herein, in particular example 15, further comprising delivering the implant via a delivery tube.

Example 20: The method of any example herein, in particular example 19, wherein the delivery tube comprises an opening and the one or more cords extend through the opening.

The above description of examples of the disclosure is not intended to be exhaustive or to limit the disclosure to the precise form disclosed above. While specific examples, and examples, are described above for illustrative purposes, various equivalent modifications are possible within the scope of the disclosure, as those skilled in the relevant art will recognize. For example, while processes or blocks are presented in a given order, alternative examples can perform routines having steps, or employ systems having blocks, in a different order, and some processes or blocks may be deleted, moved, added, subdivided, combined, and/or modified. Each of these processes or blocks can be implemented in a variety of different ways. Also, while processes or blocks are at times shown as being performed in series, these processes or blocks can instead be performed in parallel or can be performed at different times.

Certain terms of location are used herein with respect to the various disclosed examples. Although certain spatially relative terms, such as “outer,” “inner,” “upper,” “lower,” “below,” “above,” “vertical,” “horizontal,” “top,” “bottom,” and similar terms are used herein to describe a spatial relationship of one device/element or anatomical structure relative to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements, and/or steps are in any way required for one or more examples or that one or more examples necessarily include logic for deciding, with or without author input or prompting, whether these features, elements and/or steps are included or are to be performed in any particular example.

It should be understood that certain ordinal terms (e.g., “first” or “second”) can be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited. In some contexts, description of an operation or event as occurring or being performed “based on,” or “based at least in part on,” a stated event or condition can be interpreted as being triggered by or performed in response to the stated event or condition.

With respect to the various methods and processes disclosed herein, although certain orders of operations or steps are illustrated and/or described, it should be understood that the various steps and operations shown and described can be performed in any suitable or desirable temporal order. Furthermore, any of the illustrated and/or described operations or steps can be omitted from any given method or process, and the illustrated/described methods and processes can include additional operations or steps not explicitly illustrated or described.

It should be appreciated that in the above description of examples, various features are sometimes grouped together in a single example, figure, or description thereof for the purpose of streamlining the disclosure and aiding in the understanding of one or more of the various aspects of the disclosure. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the disclosure should not be limited by the particular examples described above but should be determined only by a fair reading of the claims that follow.

Unless the context clearly requires otherwise, throughout the description and the claims, the terms “comprise,” “comprising,” “have,” “having,” “include,” “including,” and the like are to be construed in an open and inclusive sense, as opposed to a closed, exclusive, or exhaustive sense; that is to say, in the sense of “including, but not limited to.”

The word “coupled,” as generally used herein, refers to two or more elements that can be physically, mechanically, and/or electrically connected or otherwise associated, whether directly or indirectly (e.g., via one or more intermediate elements, components, and/or devices. Additionally, the words “herein,” “above,” “below,” and words of similar import, when used in this application, shall refer to this application as a whole, including any disclosure incorporated by reference, and not to any particular portions of the present disclosure. Where the context permits, words in present disclosure using the singular or plural number can also include the plural or singular number, respectively.

The word “or” in reference to a list of two or more items, that word covers all of the following interpretations of the word: any of the items in the list, all of the items in the list, and any combination of the items in the list. Furthermore, as used herein, the term “and/or” used between elements (e.g., between the last two of a list of elements) means any one or more of the referenced/related elements. For example, the phrase “A, B, and/or C” means “A,” “B,” “C,” “A and B,” “A and C,” “B and C,” or “A, B, and C.”

As may be used herein, the terms “substantially” and “approximately” provide an industry-accepted tolerance for its corresponding term and/or relativity between items. For some industries, an industry-accepted tolerance is less than one percent, while for other industries, the industry-accepted tolerance can be 10 percent or more. Other examples of industry-accepted tolerances range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances can be more or less than a percentage level (e.g., dimension tolerance of less than approximately ±1%). Some relativity between items can range from a difference of less than a percentage level to a few percent. Other relativity between items can range from a difference of a few percent to magnitude of differences.

One or more examples have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks can also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules, and components herein, can be implemented as illustrated or by discrete components, application specific integrated circuits, processors executing appropriate software and the like or any combination thereof.

The one or more examples are used herein to illustrate one or more aspects, one or more features, one or more concepts, and/or one or more examples. A physical example of an apparatus, an article of manufacture, a machine, and/or of a process can include one or more of the aspects, features, concepts, examples, etc. described with reference to one or more of the examples discussed herein. Further, from figure to figure, the examples can incorporate the same or similarly named functions, steps, modules, etc. that can use the same, related, or unrelated reference numbers. The relevant features, elements, functions, operations, modules, etc. can be the same or similar functions or can be unrelated.

What is claimed is:

1. An implant for treating a heart valve comprising:
a base portion; and
a first movable arm extending from the base portion and configured to move independently of the base portion to grasp a leaflet of the heart valve.

2. The implant of claim 1, wherein the base portion has a generally curved form.

3. The implant of claim 2, wherein the first movable arm has a generally curved form that is generally opposite a curvature of the base portion.

4. The implant of claim 1, wherein the base portion comprises a network of struts forming one or more cells and wherein the first movable arm is configured to extend through at least one of the one or more cells.

5. The implant of claim 1, wherein the base portion forms a gap between ends of the base portion and wherein the first movable arm is configured to extend between the ends of the base portion.

6. The implant of claim 1, wherein the first movable arm is configured to grasp the leaflet against the base portion.

7. The implant of claim 1, further comprising a clamp extending approximately in-line with the base portion and forming a gap from the base portion.

8. The implant of claim 1, further comprising a second movable arm extending from the base portion and configured to grasp the leaflet against the first movable arm.

9. The implant of claim 1, further comprising one or more paddles extending from the base portion and configured to naturally move towards the first movable arm.

10. The implant of claim 9, further comprising one or more wires configured to hold the one or more paddles away from the first movable arm.

11. A method comprising:
grasping a leaflet of a heart valve with a first movable arm of an implant, the implant comprising a first paddle held distally from the first movable arm;
twisting the implant to cause folding of the leaflet; and
releasing the first paddle to cause the first paddle to grasp the leaflet against the first movable arm.

12. The method of claim 11, wherein the implant further comprises a second movable arm, the method further comprising grasping the leaflet between the first movable arm and the second movable arm.

13. The method of claim 11, wherein the first movable arm comprises one or more barbs configured to facilitate grasping of the leaflet.

14. The method of claim 11, wherein the implant further comprises a second paddle held distally from the first movable arm, the method further comprising releasing the second paddle to cause the second paddle to grasp the leaflet.

15. The method of claim 11, further comprising one or more cords coupled to the first paddle and configured to hold the first paddle distally from the first movable arm.

16. The method of claim 15, further comprising relaxing the one or more cords to release the first paddle.

17. The method of claim 15, wherein the one or more cords are configured to hold the first paddle at an approximately 90-degree angle relative to the first movable arm.

18. The method of claim 15, wherein the one or more cords are configured to hold the first paddle at an approximately 180-degree angle relative to the first movable arm.

19. The method of claim 15, further comprising delivering the implant via a delivery tube.

20. The method of claim 19, wherein the delivery tube comprises an opening and the one or more cords extend through the opening.
